(19) United States
(12) Patent Application Publication
(10) Pub. No.: US 2007/0219372 A1
(43) Pub. Date: Sep. 20, 2007

(54) Novel Amination Process

(75) Inventors: Mouloud Dekhane, Macclesfield Cheshire (GB); Alison Norton, Macclesfield Cheshire (GB); Anne O'Kearney-McMullan, Macclesfield Cheshire (GB); Graham Robinson, Macclesfield Cheshire (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertaje (SE)

(21) Appl. No.: 10/593,995

(22) PCT Filed: Mar. 22, 2005
(86) PCT No.: PCT/SE05/00416

§ 371 Date: Sep. 22, 2006

(30) Foreign Application Priority Data
Mar. 23, 2004 (SE) 0400759-7

(51) Int. Cl. C07D 241/02

(52) U.S. Cl. 544/376

(57) ABSTRACT

Provided herein are novel processes for the preparation of intermediates that may be useful in the preparation of 5-Ht_{1b} receptor antagonist useful in the treatment of depression, anxiety and other related diseases. The present processes may offer improved yields, purity, ease of preparation and isolation of intermediates and final product and more industrially useful reaction conditions and workability.

### FIELD OF INVENTION

The present invention provides novel processes for the preparation of a compound having the general formula I: 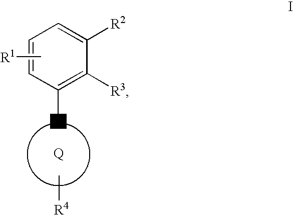
as defined herein.

### BACKGROUND OF THE INVENTION

The present invention relates to the preparation of compounds that may be useful manufacture of potentially potent orally active 5-Ht_{1b} receptor antagonist, useful in the treatment of depression, anxiety and other related diseases. An example of such a preparation is as follows: 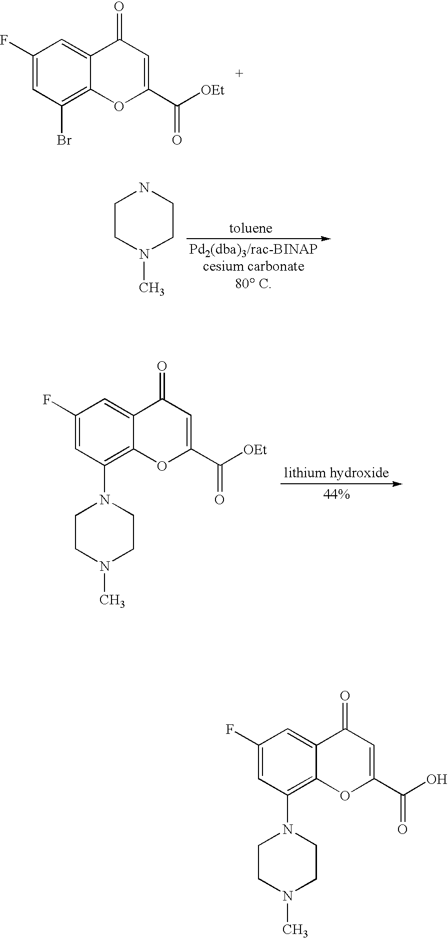
While this preparation allows for the amination of the bromoester it suffers from several drawbacks making its viability for commercial production doubtful.

Applicants provide processes for the preparation of compounds of formula I that unexpectedly and surprisingly provide improvements in product yield and process time. Also, applicants provide processes that may be substantially free of by-products and impurities. For example, the applicants' processes for the preparation of compounds of formula I do not exhibit any reversible ring opening of the chromone ring in the presence of N-methylpiperazine. Further, using the applicants' processes the desired piperazine acid is not likely to be converted into an undesirable hydrated form in the presences of aqueous alkali.

In view of the prior art it is clear that improved processes are needed for the preparation of compounds having the general formula I. Such improvements may include, for example, increased product yields, use of lower cost starting materials, lowered energy consumption, reduction in the number of synthetic steps, improved scale up conditions, and the like. The methods and compositions of the present invention are directed to these as well as other important needs, such as novel intermediates.

### DESCRIPTION OF THE INVENTION

Provided herein is a process for preparing an arylamine of formula I: 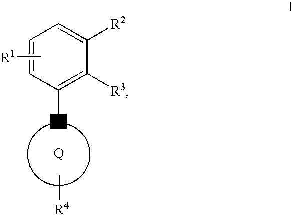
comprising heating a heterocyclyl ring moiety with an aromatic compound with a base and a solvent in the presence of a transition metal catalyst including a phosphine ligand at a temperature between about 120 and about 150° C. and for a time effective to give an arylamine compounds of formula I.
wherein:
R¹ is selected from H, C₁₋₁₀alkyl, halogen, amino, methoxy, ethoxy, cyano or hydroxy;
R² is selected from H, C₁₋₁₀alkyl, C₂₋₁₀alkenyl, C₂₋₁₀alkynyl, C₁₋₁₀alkyl-amino, C₁₋₁₀alkyl-carbonyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₆heteroaryl, C₆₋₁₀aryl or C₆₋₁₀aryl-C₁₋₆alkyl, optionally substituted by one or more groups selected from H, C₁₋₁₀alkyl, halogen, amino, methoxy, ethoxy, oxo and hydroxy;
R³ is selected from H, hydroxy, C₁₋₁₀alkyl, C₂₋₁₀alkenyl, C₂₋₁₀alkynyl, C₁₋₁₀alkyl-amino, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₆heteroaryl, C₆₋₁₀aryl or C₆₋₁₀aryl-C₁₋₆alkyl, optionally substituted by one or more groups selected from H, C₁₋₁₀alkyl, halogen, amino, methoxy, ethoxy, oxo and hydroxy;
R² and R³ can form a substituted or unsubstituted 5- or 10-membered aromatic or heteroaromatic ring having 0, 1, or 2 nitrogen atoms, 0 or 1 oxygen atoms, and 0 or 1 sulfur atoms said aromatic or heteroaromatic rings or ring systems, when substituted, having substituents selected from H, C₁₋₁₀alkyl, methoxy-C₁₋₆alkyl, oxo, halogen, amino, carbonyl, C₁₋₁₀alkyl-carbonyl, hydroxycarbony, C₁₋₆alkyl-oxycarbonyl, methoxy, ethoxy, and hydroxy,
Q is heterocyclyl ring moiety, and
R⁴ is selected from H, C₁₋₁₀alkyl, halogen, amino, methoxy, ethoxy, and hydroxy.

In another embodiment, R¹ is, independently, H, C₁-C₆ alkyl or halogen. In a more particular embodiment R¹ is, independently, hydrogen or fluoro.

In another embodiment, R² is selected from H, C₁₋₁₀alkyl, C₂₋₁₀alkenyl, C₂₋₁₀alkynyl, C₁₋₁₀alkyl-amino, C₁₋₁₀alkyl-carbonyl. In a more particular embodiment, R² may be C₁₋₆alkyl-carbonyl. In particular, R² may be methylcarbonyl.

In another embodiment, R³ is selected from H, hydroxy, C₁₋₁₀alkyl, C₂₋₁₀alkenyl, C₂₋₁₀alkynyl, C₁₋₁₀alkyl-amino, C₁₋₁₀alkyl-carbonyl. In a more particular embodiment, R³ may be hydroxy.

In another embodiment R² and R³ can form a substituted or unsubstituted 3,4-dihydro-2H-pyran ring having substitutents, independently selected from H, halogen, C₁₋₆alkyl, methoxy, ethoxy, oxo, C₁₋₃alkyl-oxycarbonyl and hydroxycarbonyl.

In another embodiment R⁴ is, independently, H, C₁-C₆ alkyl or halogen. In a more particular embodiment, R² may be methyl.

In another embodiment, Q is selected from piperidinyl, piperazinyl, morpholinyl, pyrrolidinyl, azetidinyl or isoxazolidinyl. In a more particular embodiment, Q may be piperazinyl.

The above process may be conducted under conditions and for a time effective to provide compound I. A number of different bases, solvents and catalyst may be used in the above process. The above process may further include a step for separating, filtering or washing compounds that may be carried out in any number of ways known in the art.

In another embodiment the solvent has a boiling point range between about 120 and about 150° C. In a more particular embodiment, the solvent may be an aprotic solvent selected from anisole or xylene. In particular, the solvent may be anisole.

In a more particular embodiment, the above process may be conducted at a temperature between about 125 and about 130° C. for about 1 to 8 hours.

In another embodiment, the transition metal catalyst may be selected from the late transition metals in Groups 8 through 10 of the periodic table. For example, suitable metals include platinum, palladium, iron, nickel, ruthenium and rhodium. In a more particular embodiment, the transition metal catalyst may be selected from soluble complexes of palladium or palladium acetate. In Particular, the transition metal catalyst may selected from Pd(dba)₃ or Pd₂(dba)₃. Most particularly, the transition metal catalyst may be Pd₂(dba)₃.

In another embodiment, the transition metal catalyst may include one or more phosphine ligands that influence the stability and electronic properties of the transition metal catalyst. The phosphines can be monodentate phosphine ligands or bidentate phosphine ligand. In a more particular embodiment, the phosphine ligand is a bidentate phosphine ligand. For example, suitable bidentate phosphine ligands may be selected from bidentate phosphine ligands include 2,2′-bis(diphenylphosphino)-1,1′-binaphthyl (BINAP), 1,2-bis(dimethylphosphino)ethane, 1,2-bis(diethylphosphino)ethane, 1,2-bis(dipropylphosphino)ethane, 4,5-bis(diphenylphosphino)-9,9-dimethylxanthene (xant-phos), 1,1′-bis(diphenylphosphino)ferrocene (dppf), bis(2-(diphenylphosphino)phenyl)ether [DPE-phos], 1,2-bis(diisopropylphosphino)ethane, 1,2-bis(dibutyl-phosphino)ethane, 1,2-bis(dicyclohexylphosphino)ethane, 1,3-bis(dicyclohexylphosphino)propane, 1,3-bis(diisopropylphosphino)propane, 1,4-bis(diisopropylphosphino)-butane and 2,4-bis(dicyclohexylphosphino)pentane. In particular, the phosphine ligand may be racemic 2,2′-bis(diphenylphosphino)-1,1′-binaphthyl (rac-BINAP).

In another embodiment, the base may be selected from alkoxides, alkali metal amides, alkali metal bis(trialkyl-silyl)amides, a tertiary amine, alkali, alkaline earth carbonate, bicarbonate or hydroxide. In a more particular embodiment, the base may be cesium carbonate.

In another embodiment, the catalyst may be dissolved in the solvent before being added to the suspension containing the solvent and the base.

In another embodiment, the heterocyclyl ring moiety may be added to the mixture in six portions over a 90 minute period of time.

In another embodiment, the present invention provides process for preparing compounds of formula II: 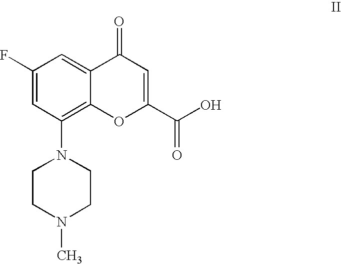
comprising:
A) heating a mixture of a compound of formula VI: 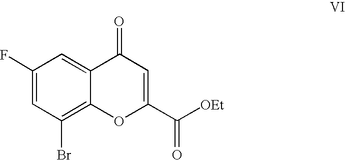
and a compound of formula VIa: 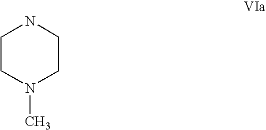
with a base and a solvent in the presence of a metal transition catalyst including a phosphine ligand at a temperature between about 120 and about 150° C. and for a time effective to give compounds of formula VIb: 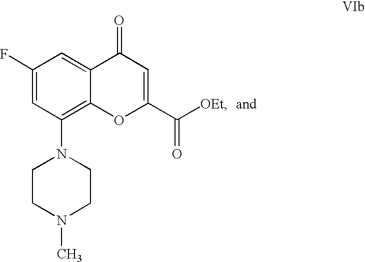
B) hydrolysis of compound of formula VIb under either basic or acidic conditions at a temperature and for a time effective to give compounds of formula (II).

The above process of preparing compounds of formula II may be conducted under conditions and for a time effective to provide compound I. A number of different bases, solvents and catalyst may be used in the above process. The above process may further include a step for separating, filtering or washing compounds that may be carried out in any number of ways known in the art.

In another embodiment the solvent has a boiling point range between about 120 and about 150° C. In a more particular embodiment, the solvent may be an aprotic solvent selected from anisole or xylene. In particular, the solvent may be anisole.

In a more particular embodiment, the above process may be conducted at a temperature between about 125 and about 130° C. for about 1 to 8 hours.

In another embodiment, the transition metal catalyst may be selected from the late transition metals in Groups 8 through 10 of the periodic table. For example, suitable metals include platinum, palladium, iron, nickel, ruthenium and rhodium. In a more particular embodiment, the transition metal catalyst may be selected from soluble complexes of palladium or palladium acetate. In Particular, the transition metal catalyst may selected from Pd(dba)₃ or Pd₂(dba)₃. Most particularly, the transition metal catalyst may be Pd₂(dba)₃.

In another embodiment, the transition metal catalyst may include one or more phosphine ligands that influence the stability and electronic properties of the transition metal catalyst. The phosphines can be monodentate phosphine ligands or bidentate phosphine ligand. In a more particular embodiment, the phosphine ligand may be a bidentate phosphine ligand. For example, suitable bidentate phosphine ligands may be selected from bidentate phosphine ligands include 2,2′-bis(diphenylphosphino)-1,1′-binaphthyl (BINAP), 1,2-bis(dimethylphosphino)ethane, 1,2-bis(diethylphosphino)ethane, 1,2-bis(dipropylphosphino)ethane, 4,5-bis(diphenylphosphino)-9,9-dimethylxanthene (xant-phos), 1,1′-bis(diphenylphosphino)ferrocene (dppf), bis(2-(diphenylphosphino)phenyl)ether [DPE-phos], 1,2-bis(diisopropylphosphino)ethane, 1,2-bis(dibutyl-phosphino)ethane, 1,2-bis(dicyclohexylphosphino)ethane, 1,3-bis(dicyclohexylphosphino)propane, 1,3-bis(diisopropylphosphino)propane, 1,4-bis(diisopropylphosphino)-butane and 2,4-bis(dicyclohexylphosphino)pentane. In particular, the phosphine ligand may be racemic 2,2′-bis(diphenylphosphino)-1,1′-binaphthyl (rac-BINAP).

In another embodiment, it may be necessary to include a suitable base. For example, suitable bases may be selected from alkoxides, alkali metal amides, alkali metal bis(trialkyl-silyl)amides, a tertiary amine, alkali, alkaline earth carbonate, bicarbonate or hydroxide. In a more particular embodiment, the base may be cesium carbonate.

In another embodiment, the catalyst may be dissolved in the solvent before being added to the suspension containing the solvent and the base.

In another embodiment, the heterocyclyl ring moiety may be added to the mixture in six portions over a 90 minute period of time.

In another embodiment, the hydrolysis of formula VIb may be carried out using aqueous sodium hydroxide at a temperature and for a time effective to give compounds of formula I. In a more particular embodiment, the temperature may be between about 45 and about 50° C. for a time between about 1 to 6 hours. The use of sodium hydroxide allows for complete hydrolysis in a reasonable timescale and at low temperatures with little or no formation of unwanted hydrated compounds. In a more particular embodiment, the hydrolysis may be carried out using a reduced charge of sodium hydroxide, such as 1.3 mol equivalents.

In another embodiment, activated carbon may be added at a temperature for a period of time to remove palladium. In particular, carbon Norit SX may be added to the mixture during hydrolysis at a temperature between about 45 and about 50° C. for a time between about 1 to 2 hours.

In another embodiment, the basic hydrolysis of formula VIb may be followed by acidifying the aqueous phase with concentrated hydrochloric acid.

In another embodiment, the acidic hydrolysis may be carried out by combining a solution of concentrated sulphuric acid and water. In particular, the mixture may be heated at a temperature between about 80 and about 100° C. for a time between about 1 to 4 hours.

In another embodiment, the pH may be adjusted. In a more particular embodiment, the pH may be adjusted to about 7.

In another embodiment, the present invention provides process for preparing compounds of formula II, as defined above, comprising:
A) heating a mixture of a compound of formula Va: 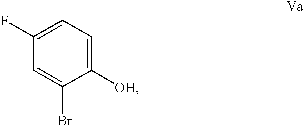
and acetyl chloride in the presence of a Lewis acid catalyst at a temperature and for a time effective to give compounds of formula Vb: 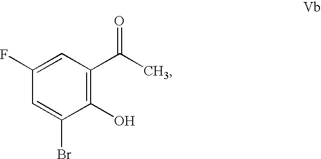
B) combining the compounds of formula Vb and ethyl oxalate to an alcohol solution at a temperature and for a time effective to give compounds of formula Vc: 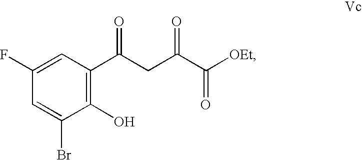
C) heating the compound of formula Vc with a mixture of acids at a temperature and for a time effective to give compounds of formula Vd: 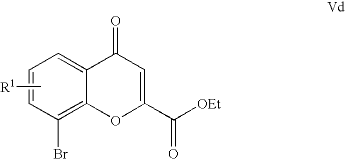
D) heating a mixture of a compound of formula Vd and a compound of formula VIa: 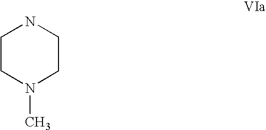
with a base and a solvent in the presence of a metal transition catalyst including a phosphine ligand at a temperature and for a time effective to give compounds of formula VIb: 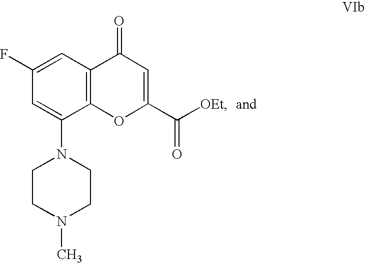
B) hydrolysis of compound of formula VIb under either basic or acidic conditions at a temperature and for a time effective to give compounds of formula (II).

If necessary a number of different bases, solvents and catalyst may be used in the process to prepare compounds of formula II. In another embodiment, the process may further include a step for separating, filtering or washing compounds that may be carried out in any number of ways known in the art.

In another embodiment, heating the compound of formula Va, acetyl chloride and the catalyst may be conducted at a temperature and for a time effective to provide compound Vb. In a more particular embodiment, the heating of formula Va, acetyl chloride and catalyst may be conducted at a temperature between about 130 and about 135° C. for about 1 to 2 hours.

In another embodiment, suitable Lewis acid catalysts include aluminum chloride, zirconium tetrachloride, Bromide tetrachloride, HF and phosphoric acid, HF. In a more particular embodiment, the Lewis acid catalyst may be selected from aluminum chloride or zirconium tetrachloride. In particular, the Lewis acid catalyst may be aluminum chloride. In a more particular embodiment the catalyst may be added in two portions.

In another embodiment, the pH level may be adjusted. In particular, the pH may be adjusted to about 7.

In another embodiment, combining the compound of formula Vb and ethyl oxalate in an alcohol solution may be conducted at a temperature and for a time effective to provide compound Vc. In a more particular embodiment, the reaction may be conducted at a temperature between about 55 and about 60° C. for a time between about 1 to 2 hours.

In another embodiment, the alcohol solution comprises sodium alkoxide in absolute alcohol. For example, suitable sodium alkoxides include sodium methoxide, sodium ethoxide sodium isopropoxide and sodium tert-butoxide. For example, suitable absolute alcohols include methanol, ethanol, propanol, butanol, isobutanol and tert-butanol. In particular, the alcohol solution comprises sodium ethoxide in absolute ethanol.

In another embodiment, reacting the compound of formula Vc with a mixture of acid may be conducted at a temperature and for a time effective to provide compound I. In a more particular embodiment, the reaction may be conducted at a temperature between about 70 and about 80° C. for a time between about 1 to 2 hours.

In another embodiment, the mixture of acids may be a mixture of acetic and hydrochloric acid.

In another embodiment, D may be carried out in solution comprising a solvent. In another embodiment the solvent has a boiling point range between about 120 and about 150° C. In a more particular embodiment, the solvent may be an aprotic solvent selected from anisole or xylene. In particular, the solvent may be anisole.

In a more particular embodiment, D may be conducted at a temperature between about 125 and about 130° C. for about 1 to 8 hours.

In another embodiment, the transition metal catalyst may be selected from the late transition metals in Groups 8 through 10 of the periodic table. For example, suitable metals include platinum, palladium, iron, nickel, ruthenium and rhodium. In a more particular embodiment, the transition metal catalyst may be selected from soluble complexes of palladium or palladium acetate. In Particular, the transition metal catalyst may selected from Pd(dba)₃ or Pd₂(dba)₃. Most particularly, the transition metal catalyst may be Pd₂(dba)₃.

In another embodiment, the transition metal catalyst may include one or more phosphine ligands that influence the stability and electronic properties of the transition metal catalyst. The phosphines can be monodentate phosphine ligands or bidentate phosphine ligand. In a more particular embodiment, the phosphine ligand may be a bidentate phosphine ligand. For example, suitable bidentate phosphine ligands may be selected from bidentate phosphine ligands include 2,2′-bis(diphenylphosphino)-1,1′-binaphthyl (BINAP), 1,2bis(dimethylphosphino)ethane, 1,2-bis(diethylphosphino)ethane, 1,2-bis(dipropylphosphino)ethane, 4,5-bis(diphenylphosphino)-9,9-dimethylxanthene (xant-phos), 1,1′-bis(diphenylphosphino)ferrocene (dppf), bis(2-(diphenylphosphino)phenyl)ether [DPE-phos], 1,2-bis(diisopropylphosphino)ethane, 1,2-bis(dibutyl-phosphino)ethane, 1,2-bis(dicyclohexylphosphino)ethane, 1,3-bis(dicyclohexylphosphino)propane, 1,3-bis(diisopropylphosphino)propane, 1,4-bis(diisopropylphosphino)-butane and 2,4-bis(dicyclohexylphosphino)pentane. In particular, the phosphine ligand may be racemic 2,2′-bis(diphenylphosphino)-1,1′-binaphthyl (rac-BINAP).

In another embodiment, in D it may be necessary to include a suitable base. For example, suitable bases may be selected from alkoxides, alkali metal amides, alkali metal bis(trialkyl-silyl)amides, a tertiary amine, alkali, alkaline earth carbonate, bicarbonate or hydroxide. In a more particular embodiment, the base may be cesium carbonate.

In another embodiment, in D the catalyst may be dissolved in the solvent before being added to the suspension containing the solvent and the base.

In another embodiment, the heterocyclyl ring moiety may be added to the mixture in six portions over a 90 minute period of time.

In another embodiment, the hydrolysis of formula VIb may be carried out using aqueous sodium hydroxide at a temperature and for a time effective to give compounds of formula I. In a more particular embodiment, the temperature may be between about 45 and about 50° C. for a time between about 1 to 6 hours. The use of sodium hydroxide allows for complete hydrolysis in a reasonable timescale and at low temperatures with little or no formation of unwanted hydrated compounds. In a more particular embodiment, the hydrolysis may be carried out using a reduced charge of sodium hydroxide, such as 1.3 mol equivalents.

In another embodiment, activated carbon may be added at a temperature for a period of time to remove palladium. In particular, carbon Norit SX may be added to the mixture during hydrolysis at a temperature between about 45 and about 50° C. for a time between about 1 to 2 hours.

In another embodiment, the basic hydrolysis of formula VIb may be followed by acidifying the aqueous phase with concentrated hydrochloric acid.

In another embodiment, the acidic hydrolysis may be carried out by combining a solution of concentrated sulphuric acid and water. In particular, the mixture may be heated at a temperature between about 80 and about 100° C. for a time between about 1 to 4 hours.

In another embodiment, the pH may be adjusted. In a more particular embodiment, the pH may be adjusted to about 7.

Finally, in another embodiment the present invention provides a compounds of the formula (IV): 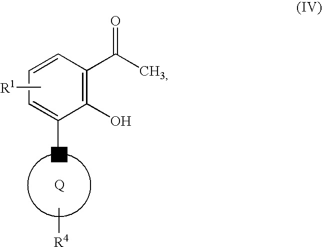
as defined above.

The above compound, R¹ is, independently, H, C₁-C₆ alkyl or halogen. In a more particular embodiment R¹ is, independently, hydrogen or fluoro.

The above compound, Q is heterocyclyl ring moiety. In another embodiment, Q is selected from piperidinyl, piperazinyl, morpholinyl, pyrrolidinyl, azetidinyl or isoxazolidinyl. In a more particular embodiment Q is piperazinyl.

The above compound, R⁴ is selected from H, C₁-C₆ alkyl, C₃-C₆ cycloalkyl, hydroxy, methoxy, aryl or heterocyclyl. In another embodiment R² is, independently, H or C₁-C₄ alkyl. In a more particular embodiment R² is methyl.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “alkyl” used alone or as a suffix or prefix, refers to monovalent straight or branched chain hydrocarbon radicals comprising 1 to about 12 carbon atoms. Unless otherwise specified, “alkyl” generally includes both saturated alkyl and unsaturated alkyl.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween about. Heterocycle may have aromatic character or may not have aromatic character.

The term “heterocyclyl” used alone or as a suffix or prefix, refers a radical derived from a heterocycle by removing at least one hydrogen from a carbon of a ring of the heterocycle.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between about two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

When a first group, structure, or atom is “directly connected” to a second group, structure or atom, at least one atom of the first group, structure or atom forms a chemical bond with at least one atom of the second group, structure or atom.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

“Saturated carbon” means a carbon atom in a structure, molecule or group wherein all the bonds connected to this carbon atom are single bond. In other words, there is no double or triple bonds connected to this carbon atom and this carbon atom generally adopts an sp³ atomic orbital hybridization.

“Unsaturated carbon” means a carbon atom in a structure, molecule or group wherein at least one bond connected to this carbon atom is not a single bond. In other words, there is at least one double or triple bond connected to this carbon atom and this carbon atom generally adopts a sp or sp² atomic orbital hybridization.

The processes and synthetic methods described hereinthroughout may be carried out in any suitable solvent. Generally, suitable solvents are solvents which are substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction, suitable solvents for a particular work-up following the reaction may be selected from known protic or aportic solvents.

Protic solvents include, for example, water and alcohols such as methanol, ethanol, propanols, including n-propanol and isopropanol, butanols, including 1-butanol, 2-butanol, i-butanol, and t-butanol, substituted ethanols, including 2-nitroethanol, 2-fluoroethanol, 2,2,2-trifluoroethanol, 2-methoxyethanol and 2-ethoxyethanol, polyols, including ethylene glycol and diethylene glycol, pentanols, including 1-, 2-, or 3-pentanol, neo-pentanol, and t-pentanol, ethers, including monomethyl ether and diethylene glycol monoethyl ether, cyclic alcohols, including cyclohexanol, aromatic alcohols, including benzyl alcohol and phenol, and glycerol, to name a few.

Aprotic solvents include, for example, hydrocarbon solvents, and halogenated derivatives thereof, such as cyclohexane, pentane, toluene, benzene, cycloheptane, methylcyclohexane, ethylbenzene, m-, o-, or p-xylene, octane, indane, nonane, and the like. Aprotic solvents further include ethers, such as diethyl ether, dimethoxymethane, tetrahydrofuran (THF), 1,3-dioxane, 1,4-dioxane, furan, ethylene glycol dimethyl ether, ethylene glycol diethyl ether, diethylene glycol dimethyl ether, diethylene glycol diethyl ether, tricithylene glycol diisopropyl ether, anisole, or t-butylmethyl ether. Other aprotic solvents include, for example, dichloromethane, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methyl-pyrrolidinone (NMP), formamide, N-methylacetamide, N-methylformamide, acetonitrile (MeCN), dimethylsulfoxide (DMSO), propionitrile, ethyl formate, methyl acetate, hexachloroacetone, acetone, ethyl methyl ketone, ethyl acetate, isopropyl acetate, t-butyl acetate, sulfolane, N,N-dimethylpropionamide, nitromethane, nitrobenzene, and hexamethylphosphoramide.

The process provided herein may provide an yield from 40-50%, 40-60% and more particularly >60%. Reaction time may also be carried at out 6-7 hours. The process provided herein may also provide a compound of Formula I substantially free of by-products and, impurities. More particularly, a compound of Formula I may be provided with <2% total organic impurities. The process provided herein also provides a compound of Formula I which does not exhibit any reversible ring opening of the chromone ring in the presence of n-methylpiperazine. Further, using the applicants process the desired piperazine acid may not be converted into undesirable hydrated forms in the presences of aqueous alkali. More particularly, a product made according to the process presented herein for example provides <3%, more particularly <2% hydrated forms of a compound of Formula I.

The examples provided below are illustrative only and are not intended to limit the scope of any claim presented herein.

### EXAMPLES

### Preparation of 1-(3-bromo-5-fluoro-2-hydroxyphenyl)ethanone

A mixture of 2-bromo-4-fluorophenol (64.0 kg, 1.00 mol equiv) and acetyl chloride (62.5 kg, 2.39 mol equiv) was stirred and heated at 50-55° C. for one hour. Excess acetyl chloride (10.8 kg) was removed by distillation and the residue was cooled to 25-30° C. then diluted with dichloromethane (120 L). The mixture was further cooled to 10-15° C. and aluminum chloride (51.0 kg, 1.15 mol equiv) was added in two portions. The temperature of the mixture was raised to 130-135° C. over one hour during which time dichloromethane (80 L) was removed by distillation. The mixture was maintained at 130-135° C. for one hour, diluted with xylene (250 L) and cooled to 10-15° C. The reaction mixture was added to a solution of 30% w/w hydrochloric acid (25 L) in water (500 L). The layers were separated and the organic phase was extracted with 10% w/w sodium hydroxide solution (300 L). The aqueous extract was cooled to 10-15° C. and adjusted to pH 6.8-7.2 with 30% w/w hydrochloric acid (55.0 kg). The solid was filtered off, washed with water (60 L), then with petroleum ether (100 L) and dried at 55-60° C. under vacuum. The yield of 1-(3-bromo-5-fluoro-2-hydroxyphenyl)ethanone was 46.0 kg (60%).

### Preparation of ethyl 8-bromo-6-fluoro-4-oxo-4H-chromene-2-carboxylate

A mixture of 1-(3-bromo-5-fluoro-2-hydroxyphenyl)ethanone (46.0 kg, 1.00 mol equiv) and diethyl oxalate (172.0 kg, 6.00 mol equiv) was added to a solution of sodium ethoxide (66.8 kg, 4.9 mol equiv) in absolute ethanol (250 L) at 60° C. The mixture was stirred at 55-60° C. for one hour and ethanol was removed by distillation. The residual mixture was diluted with water (300 L) and the precipitated solid isolated by filtration. This solid was heated with a mixture of acetic acid (210 L) and 30 w/w hydrochloric acid (55.5 L) at 70-80° C. for two hours. After cooling at 25° C., the mixture was diluted with water (150 L and 100 L), then with 12% w/w sodium bicarbonate solution (50 L) and finally methanol (100 L) before drying at 70° C. under vacuum. The yield of ethyl 8-bromo-6-fluoro-4-oxo-4H-chromene-2-carboxylate was 38.6 kg (63%).

### Preparation of 6-fluoro-8-(4-methylpiperazin-1-yl)-4-oxo-4H-chromene-2-carboxylic acid

(Basic Hydrolysis): The reactors required for the amination stage were carefully dried by rinsing with acetone and heating to 110° C. under vacuum; they were then rinsed with anisole under partial reflux. An inert atmosphere was maintained during all charging operations by pressurizing the reactors with nitrogen (two cycles) then evacuating and releasing the vacuum with nitrogen (two cycles). A mixture of tris(dibenzylideneacetone)dipalladium (2.25 kg, 0.02 mol equiv), rac-BINAP (3.06 kg, 0.04 mol equiv) and anisole (135 L) was adjusted to 25° C. and added to a stirred suspension of cesium carbonate (56.0 kg, 1.40 mol equiv) in anisole (230 L) at 25° C. followed by a line wash in anisole (19.5 L). The mixture was heated to 125° C. A solution of ethyl 8-bromo-6-fluoro-4-oxo-4H-chromene-2-carboxylate (38.7 kg, 1.00 mol equiv) and N-methylpiperazine (13.5 kg, 1.10 mol equiv in anisole (154 L) maintained at 45° C. was added in six portions over 90 minutes to the heated catalyst mixture; this was followed by a line wash of anisole (38.4 L). The resulting mixture was maintained at 125° C. for an additional 6.5 hours then cooled to 45° C. and diluted with water (233 L). 47% w/w sodium hydroxide solution (13.6 kg, 1.30 mol equiv) was added and the mixture was stirred at 45° C. for an additional 2 hours then cooled to 25° C. and filtered through Haborlite 800 filter acid. The filter cake washed with water (39 L) and anisole (50 L). After allowing the filtrate to settle, the aqueous layer was separated and diluted with tetrahydrofuran (77 L) and methanol (77 L). The solution was acidified with concentrated hydrochloric acid (38.7 kg, 3.20 mol equiv) at 20° C. The precipitated solid was filtered off, washed with a mixture of tetrahydrofuran (58 L), methanol (58 L) and water (58 L) followed by methanol (50 L) and dried at 40° C. under a flow of nitrogen to give 6-fluoro-8-(4-methylpiperazin-1-yl)-4-oxo-4H-chromene-2-carboxylic acid. The mean weight of product from three batches was 23.9 kg (corrected for strength) which represents a yield of 64%. 1H NMR (400 MHz, DMSO-d6, CF₃CO₂H) δ 2.94 (s, 3H), 3.20 (t, J=12.0 Hz, 2H), 3.32 (t, J=11.1 Hz, 2H), 3.62 (d, J=12.0 Hz, 2H), 3.90 (d, J=13.0 Hz, 2H), 6.93 (S, 1H), 7.31(dd, J=8.0, 3.0 Hz, 1H), 7.38 (DD, J=10.4, 3.0 Hz, 1H).

(Acidic Hydrolysis): The vessels required for the amination stage were carefully dried and processing was carried out under a nitrogen atmosphere. To a mixture of tris(dibenzylideneacetone)dipalladium (1.16 g, 0.02 mol equiv), rac-BINAP (1.58 g, 0.04 mol equiv) and anisole (200 mL) was added cesium carbonate (28.95 g, 1.40 mol equiv). The mixture was heated to 125° C. and a solution of ethyl 8-bromo-6-fluoro-4-oxo-4H-chromene-2-carboxylate (20.0 g, 1.00 mol equiv) and N-methylpiperazine (6.99 g, 1.10 mol equiv) in anisole (90 mL) maintained at 45° C. was added in six portions over 90 minutes; this was followed by an anisole (10 mL) line wash. The resulting mixture was maintained at 125° C. for an additional 3 hours then cooled to 75° C. and diluted with water (200 mL). A solution of concentrated sulphuric acid (55.4 g, 8.8 mol equiv) in water (100 mL) was added at 75° C. and the mixture was heated at 96° C. for 4 hours. The mixture was cooled to 75° C. and the layers separated. Anisole (100 mL) and 47% w/w sodium hydroxide solution (37.75 g, 6.99 mol equiv) were added to the hot aqueous layer and the mixture was cooled to 20° C. The pH was adjusted to 7 by adding 47% w/w sodium hydroxide solution (39.26 g, 7.29 mol equiv) and the precipitated solid was filtered off, washed with a mixture of tetrahydrofuran (30 mL), methanol (30 mL), then dried at 40° C. under vacuum. The yield of give 6-fluoro-8-(4-methylpiperazin-1-yl)-4-oxo-4H-chromene-2-carboxylic acid was 14.2 g (73%).

### Preparation of 1-[5-fluoro-2-hydroxy-3-(4-methylpiperazin-1-yl)phenyl]ethanone

The vessels required for the amination stage were carefully dried and processing was carried out under a nitrogen atmosphere.

To a mixture of tris(dibenzylideneacetone)dipalladium (1.16 g, 0.02 mol equiv), rac-BINAP (1.58 g, 0.04 mol equiv) and anisole (200 mL) was added cesium carbonate (28.95 g, 1.40 mol equiv). The mixture was heated to 125° C. and a solution of ethyl 8-bromo-6-fluoro-4-oxo-4H-chromene-2-carboxylate (20.0 g, 1.00 mol equiv) and N-methylpiperazine (6.99 g, 1.10 mol equiv) in anisole (90 mL) maintained at 45° C. was added in six portions over 90 minutes; this was followed by an anisole (10 mL) line wash. The resulting mixture was maintained at 125° C. for an additional 3 hours then cooled to 20° C. and diluted with water (120 mL). 47% w/w sodium hydroxide solution (11.88 kg, 2.20 mol equiv) was added and the mixture was stirred at 20° C. for 0.75 hours. After allowing the filtrate to settle, the aqueous layer was separated and filtered through Celite. Tetrahydrofuran (30 mL) and methanol (30 mL) were added to the filtrate and the pH was adjusted to 7. The solid piperazine acid was filtered off and the mother liquor was freeze dried to give an orange-brown solid. This solid was subjected to chromatography on silica (acetonitrile/methanol:10/00 to 9/1), yielding 1-[5-fluoro-2-hydroxy-3-(4-methylpiperazin-1-yl)phenyl]ethanone (0.72 g, 5%). 1H NMR (400 MHz, CDCl3) δ 2.54 (s, 3H), 2.61 (s, 3H), 2.89 (br s, 4H), 3.30 (br s, 4H), 6.84 (dd, J=3, 10 Hz, 1H), 7.07 (dd, J=3, 8.5 Hz, 1H)

1. A process of preparing an arylamine of formula I: 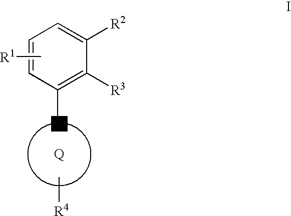
comprising heating a heterocyclyl ring moiety with an aromatic compound with a base and a solvent in the presence of a transition metal catalyst including a phosphine ligand at a temperature between about 120 and about 150° C. and for a time effective to give an arylamine compound of formula I,
wherein: R¹ is selected from H, C₁₋₁₀alkyl, halogen, amino, methoxy, ethoxy, or hydroxy;
R² is selected from H, C₁₋₁₀alkyl, C₂₋₁₀alkenyl, C₂₋₁₀alkynyl, C₁₋₁₀alkyl-amino, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₅heteroaryl, C₆₋₁₀aryl or C₆₋₁₀aryl-C₁₋₆alkyl, wherein said H, C₁₋₁₀alkyl, C₂₋₁₀alkenyl, C₂₋₁₀alkynyl, C₁₋₁₀alkyl-amino, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₅heteroaryl, C₆₋₁₀aryl or C₆₋₁₀aryl-C₁₋₆alkyl, used in defining R² is optionally substituted by one or more groups selected from H, C₁₋₁₀alkyl, halogen, amino, methoxy, ethoxy, oxo and hydroxy;
R³ is selected from H, C₁₋₁₀alkyl, C₂₋₁₀alkenyl, C₂₋₁₀alkynyl, C₁₋₁₀alkyl-amino, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₅heteroaryl, C₆₋₁₀aryl or C₆₋₁₀aryl-C₁₋₆alkyl, wherein said H, C₁₋₁₀alkyl, C₂₋₁₀alkenyl, C₂₋₁₀alkynyl, C₁₋₁₀alkyl-amino, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₁₀heterocyclyl-C₁₋₆alkyl, C₃₋₅heteroaryl, C₆₋₁₀aryl or C₆₋₁₀aryl-C₁₋₆alkyl, used in defining R³ is optionally substituted by one or more groups selected from H, C₁₋₁₀alkyl, halogen, amino, methoxy, ethoxy, oxo and hydroxy;
R² and R³ can form a substituted or unsubstituted 5- or 10-membered aromatic or heteroaromatic ring having 0, 1, 2, or 3 nitrogen atoms, 0 or 1 oxygen atoms, and 0 or 1 sulfur atoms said aromatic or heteroaromatic rings or ring systems, when substituted, having substituents selected from C₁₋₁₀alkyl, oxygen, oxo, halogen, amino, carbonyl, hydroxycarbony, C₁₋₆alkyl-oxycarbonyl, methoxy, methoxy-C₁₋₆alkyl, ethoxy, and hydroxy.
R⁴ is selected from H, C₁₋₁₀alkyl, halogen, amino, methoxy, ethoxy, and hydroxy;

2. A process according to claim 1, wherein R¹ is, independently, hydrogen or fluoro.

3. A process according to claim 1, wherein R² is methyl-carbonyl

4. A process according to claim 1, wherein R³ is hydroxy.

5. A process according to claim 1, wherein R⁴ is methyl.

6. A process according to claim 1, wherein Q is piperazinyl.

7. A process according to claim 1, wherein R² and R³ form an optionally substituted 3,4-dihydro-2H-pyran ring having substitutents, independently selected from H, oxo, C₁₋₃alkyl-oxycarbonyl and hydroxycarbonyl.

8. A process according to claim 1, wherein said base is cesium carbonate

9. A process according to claim 1, wherein said solvent is anisole.

10. A process according to claim 1, wherein said solvent is xylene.

11. A process according to claim 1, wherein said transition metal catalyst is selected from palladium or palladium acetate.

12. A process according to claim 1, wherein said transition metal catalyst is d₂(dba)₃.

13. A process according to claim 1, wherein said phosphine ligand is racemic 2,2′-bis(diphenylphosphino)-1,1′-binaphthyl (rac-BINAP).

14. A process according to claim 1, wherein said heating is at a temperature between about 125 and about 130° C.

15. A process of preparing a compound of formula II: 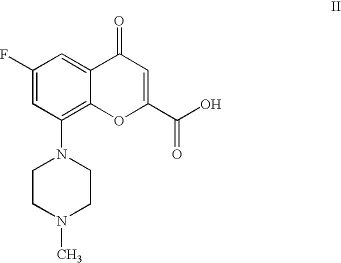
comprising:
A) heating a mixture of a compound of formula II: 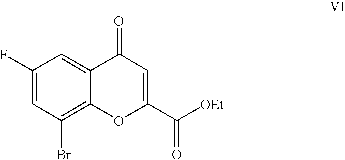
and a compound of formula VIa: 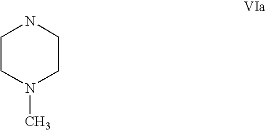
with a base and a solvent in the presence of a metal transition catalyst including a phosphine ligand at a temperature between about 120 and about 150° C. and for a time effective to give compounds of formula VIb: 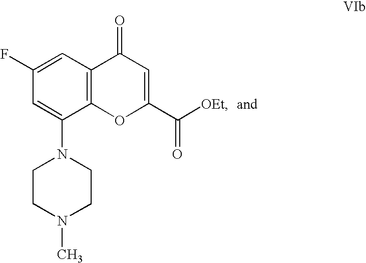
B) hydrolysis of compound of formula VIb under either basic or acidic conditions at a temperature and for a time effective to give compounds of formula (II).

16. A process according to claim 15, wherein said base is cesium carbonate

17. A process according to claim 15, wherein said solvent is anisole.

18. A process according to claim 15, wherein said solvent is xylene.

19. A process according to claim 15, wherein said transition metal catalyst is selected from palladium or palladium acetate.

20. A process according to claim 15, wherein said transition metal catalyst is Pd₂(dba)₃.

21. A process according to claim 15, wherein said phosphine ligand is racemic 2,2′-bis(diphenylphosphino)-1,1′-binaphthyl (rac-BINAP).

22. A process according to claim 15, wherein said heating is at a temperature between about 125 and 130° C.

23. A process of preparing a compound of formula II comprising:
A) heating a mixture of a compound of formula Va: 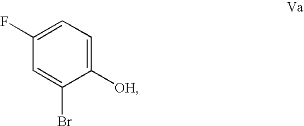
and acetyl chloride in the presence of a Lewis acid catalyst at a temperature and for a time effective to give compounds of formula Vb: 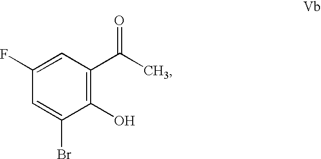
B) combining the compounds of formula Vb and diethyl oxalate to an alcohol solution at a temperature and for a time effective to give compounds of formula Vc: 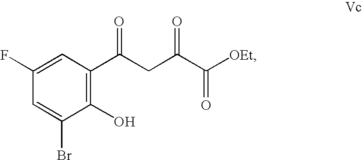
C) heating the compound of formula Vc with a mixture of acids at a temperature and for a time effective to give compounds of formula II: 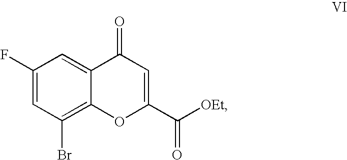
D) heating a mixture of a compound of formula II and a compound of formula VIa: 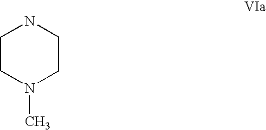
with a base and a solvent in the presence of a metal transition catalyst including a bidentate phosphine ligand at a temperature between about 120 and 150° C. and for a time effective to give compounds of formula VIb: 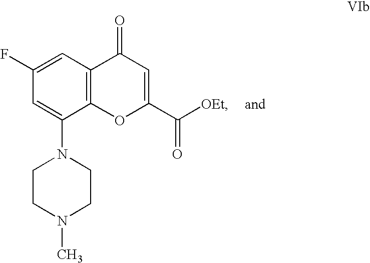
B) hydrolysis of compound of formula VIb under either basic or acidic conditions at a temperature and for a time effective to give compounds of formula (II).

24. A process according to claim 23, wherein said Lewis acid catalyst is aluminum chloride.

25. A process according to claim 23, wherein said Lewis acid catalyst is zirconium tetrachloride.

26. A process according to claim 23, wherein said alcohol solution is sodium ethoxide in absolute ethanol.

27. A process according to claim 23, wherein said mixture of acids is a mixture of acetic acid and hydrochloric acid.

28. A process according to claim 23, wherein said base is cesium carbonate

29. A process according to claim 23, wherein said solvent is anisole.

30. A process according to claim 23, wherein said solvent is xylene.

31. A process according to claim 23, wherein said transition metal catalyst is selected from palladium or palladium acetate.

32. A process according to claim 23, wherein said transition metal catalyst is Pd₂(dba)₃.

33. A process according to claim 23, wherein said phosphine ligand is racemic 2,2′-bis(diphenylphosphino)-1,1′-binaphthyl (rac-BINAP).

34. A process according to claim 23, wherein said heating is at a temperature between about 125 and 130° C.

35. A compound of the formula (IV): 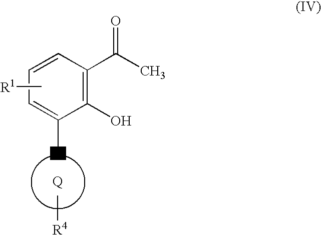
wherein
R¹ is selected from H, C₁-C₆ alkyl, halogen, hydroxy, methoxy or cyano,
Q is selected from piperidinyl, piperazinyl, morpholinyl, pyrrolidinyl, azetidinyl or isoxazolidinyl, and R⁴ is selected from H, C₁-C₆ alkyl, C₃-C₆ cycloalkyl, hydroxy, methoxy, aryl or heterocyclyl.

36. A compound according to claim 35, wherein R¹ is, independently, hydrogen or fluoro.

37. A compound according to claim 35, wherein Q is piperazinyl.

38. A compound according to claim 35, wherein R⁴ is, independently, H or C₁-C₄ alkyl.

39. A compound according to claim 35, wherein R⁴ is methyl.
